(19) United States
(12) Patent Application Publication
(10) Pub. No.: US 2014/0149050 A1
(43) Pub. Date: May 29, 2014

(54) DATA PROCESSING SYSTEM FOR CHROMATOGRAPH

(71) Applicant: Toshinobu Yanagisawa, Kyoto-shi (JP)

(72) Inventor: Toshinobu Yanagisawa, Kyoto-shi (JP)

(73) Assignee: SHIMADZU CORPORATION, Kyoto-shi, Kyoto (JP)

(21) Appl. No.: 14/131,251

(22) PCT Filed: Jun. 22, 2012
(86) PCT No.: PCT/JP2012/066068

§ 371 Date: Jan. 7, 2014

(30) Foreign Application Priority Data
Jul. 8, 2011 (JP) 2011-151433

(51) Int. Cl. G01N 33/00

(52) U.S. Cl. CPC G01N 33/00
USPC 702/23

(57) ABSTRACT

A data processing system for a chromatograph, in which, when the inflection points in one of the front and rear parts of a peak divided at a peak top cannot be appropriately detected, a tangent to the peak is drawn at the detected inflection point, and an intersection point of the tangent and a baseline is detected. Furthermore, a point at which a perpendicular drawn from the peak top to the baseline intersects with the baseline is also detected. Then, the distance between the intersection points is calculated, and a value obtained by doubling this distance is adopted as a peak width.

### TECHNICAL FIELD

The present invention relates to a data processing system for a chromatograph, such as a gas chromatograph or liquid chromatograph.

### BACKGROUND ART

The performance or efficiency of a chromatographic apparatus can be judged by various indices, such as the theoretical plate number or the resolution (the degree of separation of the peaks). The theoretical plate number is an index representing the separation performance of a column, which is calculated from the retention time and the peak width of a component on a chromatogram.

According to the United States Pharmacopeia (USP), which is published under the jurisdiction of the U.S. Food and Drug Administration (FDA), the peak width is defined as follows (Non-Patent Document 1): As shown in FIG. 1, inflection points C1 and C2 in the front and rear parts of the peak divided at the peak top P are respectively located, and a tangent to the peak curve is drawn at each of the inflection points C1 and C2. The intersection points B1 and B2 of the two tangents with the baseline BL are located, and the distance between the two intersection points B1 and B2 is adopted as the peak width W. Using this peak width W and the retention time Tr, the theoretical plate number N is given by the following equation (1):

N=16×(Tr/W)² (1)

In the Japanese Pharmacopeia (JP), which is published under the jurisdiction of the Ministry of Health, Labor and Welfare, the peak width is defined as follows: As shown in FIG. 2, a perpendicular is drawn from the peak top P downward, and the point Q at which it intersects with the baseline BL is located. A straight line passing through the point at one half of the peak height PQ and extending parallel to the baseline BL is drawn, and the distance between the points D1 and D2 at which the straight line intersects with the front and rear parts of the peak, respectively, is adopted as the peak width W_{0.5}. In this case, the theoretical plate number N is given by the following equation (2) (Non-Patent Document 2):

N=5.54×(Tr/W_{0.5})² (2)

Both equations (1) and (2) yield the same value of the theoretical plate number N if the peak shape of the chromatogram is an ideal Gaussian distribution (normal distribution).

The peak widths W and W_{0.5} are also used for calculating the resolution or other indices as well as the theoretical plate number. It is also common to calculate a peak width W_{0.5} or W_{0.1} at a height of 5% or 10% from the baseline BL to determine the symmetry factor or other indices.

Although the aforementioned calculation method according to the Japanese Pharmacopeia is intended for calculating the peak width at the 50% height level from the baseline BL, the peak widths at different heights (e.g. 5% or 10%) can also be calculated by similar procedures. Accordingly, in the following description, such methods are collectively referred to as the “peak width calculation method of the Japanese Pharmacopeia.”

### BACKGROUND ART DOCUMENT

### Non-Patent Document

Non-Patent Document 1: “Reviewer Guidance—Validation of Chromatographic Methods”, [online], Center for Drug Evaluation and Research (CDER), [searched on Jun. 11, 2012], the Internet <URL: http://www.fda.gov/downloads/Drugs/GuidanceComplianceRegulatoryInformation/Guidanc es/ucm072974.pdf>

Non-Patent Document 2: “Dai Juugo Kaisei Nihon Yakkyokuhou (The Japanese Pharmacopeia, Fifteenth Edition)”, the Ministry of Health, Labor and Welfare, [searched on Jun. 11, 2012], the Internet <URL: http://jpdb.nihs.gojp/jp15/YAKKYOKUHOU15.pdf>

### SUMMARY OF THE INVENTION

### Problem to be Solved by the Invention

Calculating a peak width in the previously described manner is frequently required in a data processing system for a chromatograph in order to compute the theoretical plate number, the degree of separation or other indices. However, when there are two adjacent peaks overlapping each other as shown in FIGS. 3 and 4, the peak width may possibly be incorrectly calculated, and in the worst case scenario, it will be impossible to calculate the peak width.

FIG. 3 illustrates an example of calculating the peak width W by the peak width calculation method of the U.S. Pharmacopeia as shown in FIG. 1. In the left-hand peak 1 of the two peaks neighboring each other in FIG. 3, the inflection points can be appropriately located in both the front and rear parts of the peak, so that the peak width W can be correctly calculated. By contrast, in the right-hand peak 2, the inflection point in the front part of the peak is detected at a position displaced from the true position, which is because the front part is overlapped with the peak 1. In such a case, the width W of the peak 2 cannot be correctly calculated.

FIG. 4 illustrates an example of calculating the peak width W_{0.5} by the peak width calculation method of the Japanese Pharmacopeia. In the peak 1 of the two peaks in FIG. 4, the points where the straight line parallel to the baseline intersects with the peak 1 can be located in both the front and rear parts of the peak, so that the peak width W_{0.5} can be correctly calculated. By contrast, the width W_{0.5} of the peak 2 cannot be calculated, since the front part of this peak is overlapped with the peak 1 and the intersection point in that part cannot be located.

In the previously described cases, it is possible to calculate the peak width after separating the peaks by deconvolution computing. However, such a method requires an additional processing time for the peak separation and yet can yield no more than a speculated value.

The problem to be solved by the present invention is to provide a data processing system for a chromatograph, which is capable of more reliably calculating a peak width, without performing complex processing, even if the point of inflection or intersection in one of the front and rear parts of the peak cannot be appropriately obtained due to an overlapping of the peak with another one.

### Means for Solving the Problem

The first mode of the data processing system for a chromatograph according to the present invention aimed at solving the previously described problem includes:

a) a baseline determiner for determining a baseline of a chromatogram;

b) a peak top detector for detecting a peak top of the chromatogram; and

c) a peak width calculator for calculating a peak width of a peak by detecting an inflection point in a front part or a rear part of the peak divided at the peak top, by detecting an intersection point at which a tangent to the peak at the inflection point intersects with the baseline as well as an intersection point at which a perpendicular drawn from the peak top to the baseline intersects with the baseline, by determining the distance between the two intersection points, and by calculating, as the peak width of the peak, a value which equals two times the aforementioned distance.

The second mode of the data processing system for a chromatograph according to the present invention aimed at solving the previously described problem includes:

a) a baseline determiner for determining a baseline of a chromatogram;

b) a peak top detector for detecting a peak top of the chromatogram; and

c) a peak width calculator for calculating a peak width of a peak by drawing a straight line parallel to the baseline at a height of M-% of the height of the peak top from the baseline, by detecting an intersection point at which the straight line intersects with the peak in a front part or a rear part of the peak divided at the peak top, by detecting an intersection point at which a perpendicular drawn from the peak top to the straight line intersects with the straight line, by determining the distance between the two intersection points, and by calculating, as the peak width of the peak, a value which equals two times the aforementioned distance.

Calculation equations for the theoretical plate number or other indices for a chromatogram are formulated on the assumption that the peak shape of the chromatogram originally follows the Gaussian distribution. If the peak shape of the chromatogram is an ideal Gaussian distribution, the front and rear parts of the peak will be symmetrical with respect to the peak top. The basic idea of the present invention is to suppose this symmetry and calculate the peak width by doubling the width of the front or rear part of the peak, whichever computable, without performing the process of separating the peaks by deconvolution computing or other calculations.

If there are no peaks overlapping each other, or if an overlapping of two peaks does not affect the calculation of the peak width as in the case of the peak 1 in FIGS. 3 and 4, it is preferable to use a conventional method, i.e. to calculate, as the peak width, the distance between the points at which the tangents to the peak at the inflection points in the front and rear parts of the peak respectively intersect with the baseline, or the distance between the two points at each of which the straight line parallel to the baseline intersects with the peak.

### EFFECT OF THE INVENTION

With the data processing system for a chromatograph according to the present invention, the width of a target peak can be easily calculated as long as the width of one of the front and rear parts of the peak can be calculated. Furthermore, if the peak shape is adequately symmetrical, the calculated peak width will be more reliable than in the case where the peaks are separated by deconvolution computing.

### BRIEF DESCRIPTION OF THE DRAWINGS

FIG. 1 is a diagram illustrating a peak width calculation method of the U.S. Pharmacopeia.

FIG. 2 is a diagram illustrating a peak width calculation method of the Japanese Pharmacopeia.

FIG. 3 is a diagram showing an example of a peak whose width is differently calculated from the actual value by the peak width calculation method of the U.S. Pharmacopeia.

FIG. 4 is a diagram showing an example of a peak whose width cannot be calculated by the peak width calculation method of the Japanese Pharmacopeia.

FIG. 5 is a schematic configuration diagram of a gas chromatograph analyzer system including one embodiment of the data processing system according to the present invention.

FIG. 6 is a flowchart showing a procedure of calculating the theoretical plate number.

FIG. 7 is a flowchart showing the procedure of the first mode of the peak width calculation process according to the present embodiment.

FIG. 8 is a diagram illustrating the first mode of the peak width calculation process.

FIG. 9 is a flowchart showing the procedure of the second mode of the peak width calculation process according to the present embodiment.

FIG. 10 is a diagram illustrating the second mode of the peak width calculation process.

### MODE FOR CARRYING OUT THE INVENTION

### Embodiment

One embodiment of the data processing system for a chromatograph according to the present invention is hereinafter described with reference to FIGS. 5-10.

FIG. 5 is a schematic configuration diagram of a gas chromatograph analyzer system including the data processing system according to the present embodiment. A liquid sample is injected through a syringe 11 or similar device into a sample vaporization chamber 12, where the sample is vaporized. The vaporized sample is carried by a stream of carrier gas supplied from a carrier gas passage 13 at a constant flow rate, to be sent into a column 14. While passing through the column 14, various components contained in the sample are temporally separated, to be eventually discharged from the column 14 and sequentially detected by a detector 15. The detection signals produced by the detector 15 are converted into digital data and then sequentially sent to the data processing system 16, in which the data are temporarily stored on a hard disk or in similar storage unit. After the analysis of one sample is completed (or after a set of analyses continuously performed for a plurality of samples are completed), the data are read from the storage unit, to be subjected to various kinds of data processing, such as a chromatogram creation or peak detection.

The substance of the data processing system 16 is a dedicated or multi-purpose computer, with a predetermined processing program running on it so as to make this computer function as a chromatogram creator 161, a baseline determiner 162, a peak detector 163, and a peak width calculator 164, and to make it perform data processing for various kinds of analyses.

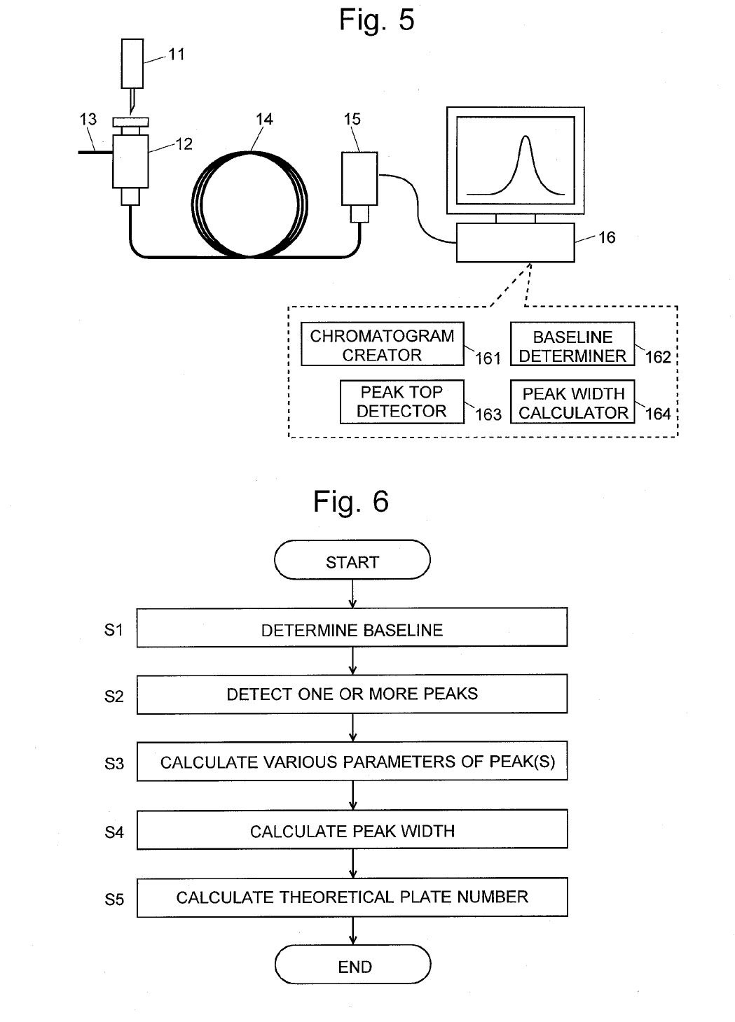

A procedure of calculating the theoretical plate number N in the data processing system 16 is shown in the flowchart of FIG. 6. For a chromatogram data read from the storage unit in the data processing system 16, a chromatogram is initially created by the chromatogram creator 161. Based on a predetermined algorithm, a baseline for the created chromatogram is determined by the baseline determiner 162, and one or more peaks are detected on the same chromatogram by the peak detector 163 (Steps S1 and S2). Then, for each and every peak, or for one or more necessary peaks, parameters characterizing the peak are calculated, such as the peak starting time Ts, peak top time Tp, peak ending time Te, peak height Hp, and peak area S (Step S3). Subsequently, the peak width is calculated by the peak width calculator 164 using the aforementioned parameters and chromatogram data (Step S4), and the theoretical plate number N is calculated by equation (1), (2) or another equation formulated for the peak width calculation method (Step S5).

The data processing according to the present invention is characterized by the peak width calculation process in Step S4. Accordingly, this process will be hereinafter described in detail. As explained earlier, there are two major methods for the peak width calculation, i.e. the method of the U.S. Pharmacopeia and the method of the Japanese Pharmacopeia. The following description initially deals with the procedure of calculating the peak width W according to the U.S. Pharmacopeia.

### [First Mode of Peak Width Calculation Process]

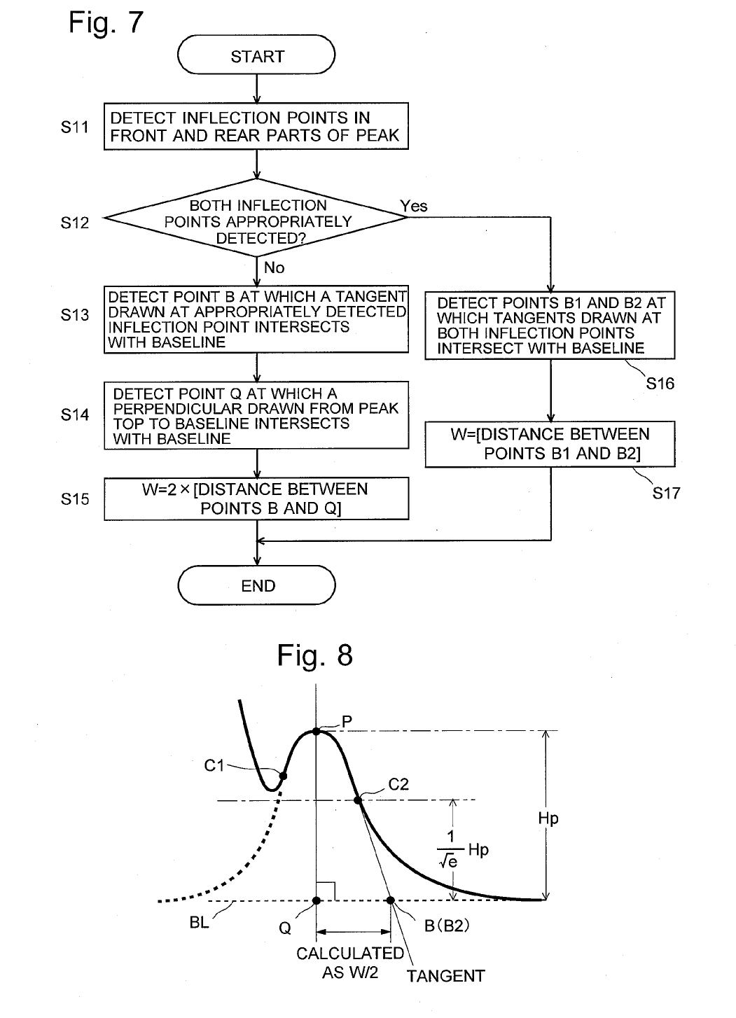

FIG. 7 is a flowchart of the first mode of the peak width calculation process according to the present embodiment. The procedure of the first mode of the peak width calculation process is hereinafter described with reference to the illustrative diagrams of FIGS. 1 and 8.

In the first mode of the peak width calculation process according to the present embodiment, a target peak spread over a range of time from its peak starting time to its peak ending time is initially searched for an inflection point in each of the front and rear parts of the peak divided at the peak top P (Step S11). A commonly used algorithm for searching for an inflection point is as follows:

Starting from the peak top P and moving backward in time, the first and second differential values are calculated at each point on the peak curve, and it is determined whether or not the second differential value is zero and the first differential value is greater than zero (i.e. a positive value). The point on the chromatogram which satisfies this condition is adopted as the inflection point Cl in the front part. The inflection point C2 in the rear part can be found in a similar way, except for the searching point moving from the peak top P forward in time and the first differential value being tested as to whether or not it is less than zero (e.g. a negative value). The search for the inflection point may be initiated from the base of the peak (the beginning and ending points of the peak) instead of the peak top P. For the calculation of the first and second differential values, commonly known algorithms can be used, such as the Savitzky-Golay method.

Subsequently, it is determined whether or not the inflection points have been appropriately detected in both the front and rear parts of the peak (Step S12). It is known that, if the peak shape is an ideal Gaussian distribution, the inflection point is located at a height equal to 1/e^{0.5} times the peak height Hp, where e is the base of the natural logarithm (see JP-A 2004-184148). Accordingly, for example, whether or not the detected inflection points are appropriate can be determined by examining whether or not the heights of the detected inflection points from the baseline are within a predetermined range from 1/e^{0.5} Hp,

In Step S12, if it has been confirmed that the inflection points C1 and C2 have been appropriately detected in both the front and rear parts of the peak as in FIG. 1, a tangent to the peak is drawn at each of the inflection points C1 and C2, using the first differential value, and the points B1 and B2 at which the tangents respectively intersect with the baseline are located (Step S15). Eventually, the distance between the two intersection points B1 and B2 is adopted as the peak width W (Step S16). These calculation steps are in accordance with the conventional method.

On the other hand, as in FIG. 8, when the inflection point in one of the front and rear parts of the peak has not been appropriately detected, it is impossible to correctly calculate the peak width W by the conventional method. In the first mode of the peak width calculation process according to the present embodiment, such a case is handled as follows: A tangent to the peak is drawn at the inflection point which has been appropriately detected (the point C2 in the example of FIG. 8), and the intersection point B (or B2) of the tangent and the baseline BL is detected. The point Q at which a perpendicular drawn from the peak top P to the baseline BL intersects with the baseline BL is also detected (Steps S13 and S14). The distance between the two intersection points B and Q detected in Steps S13 and S14 is calculated, and the value obtained by doubling this distance is adopted as the peak width W (Step S15).

Thus, even if one of the inflection points cannot be appropriately detected, the peak width W can be calculated by the processes of Steps S13-S15.

### [Second Mode of the Peak Width Calculation Process]

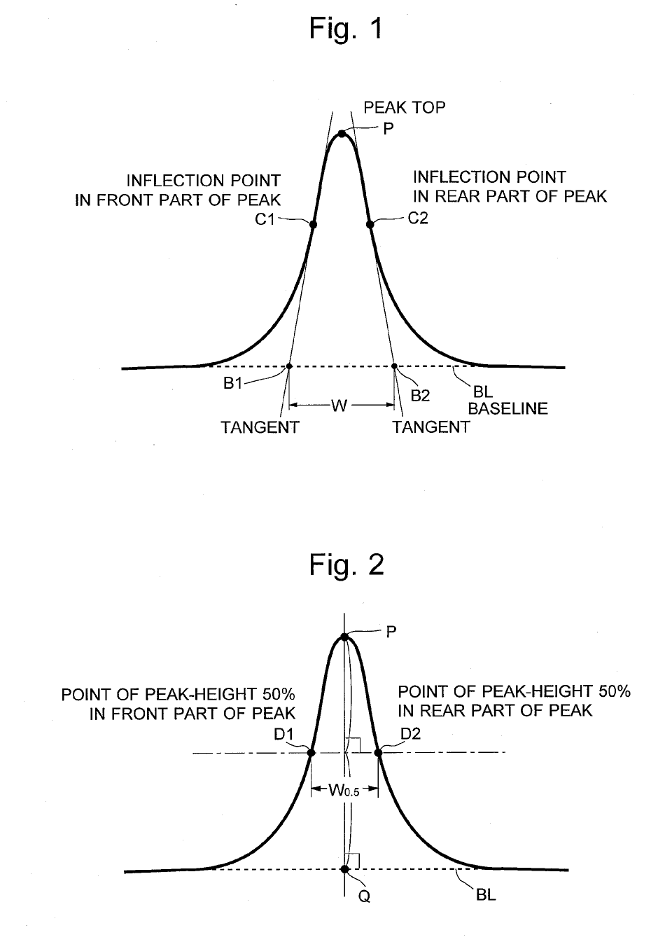

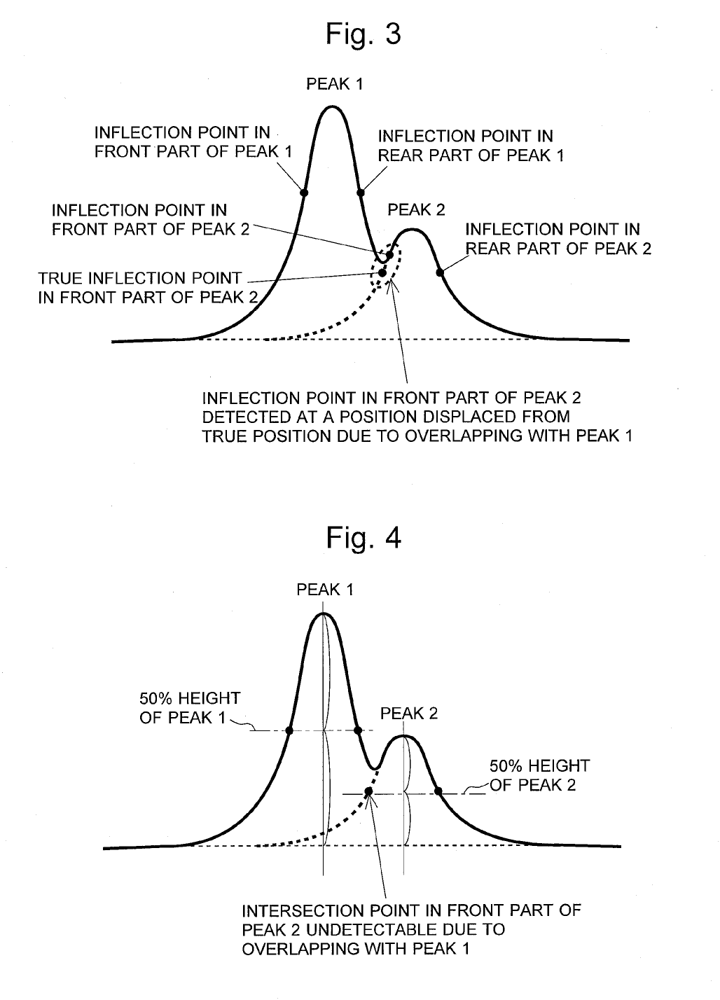

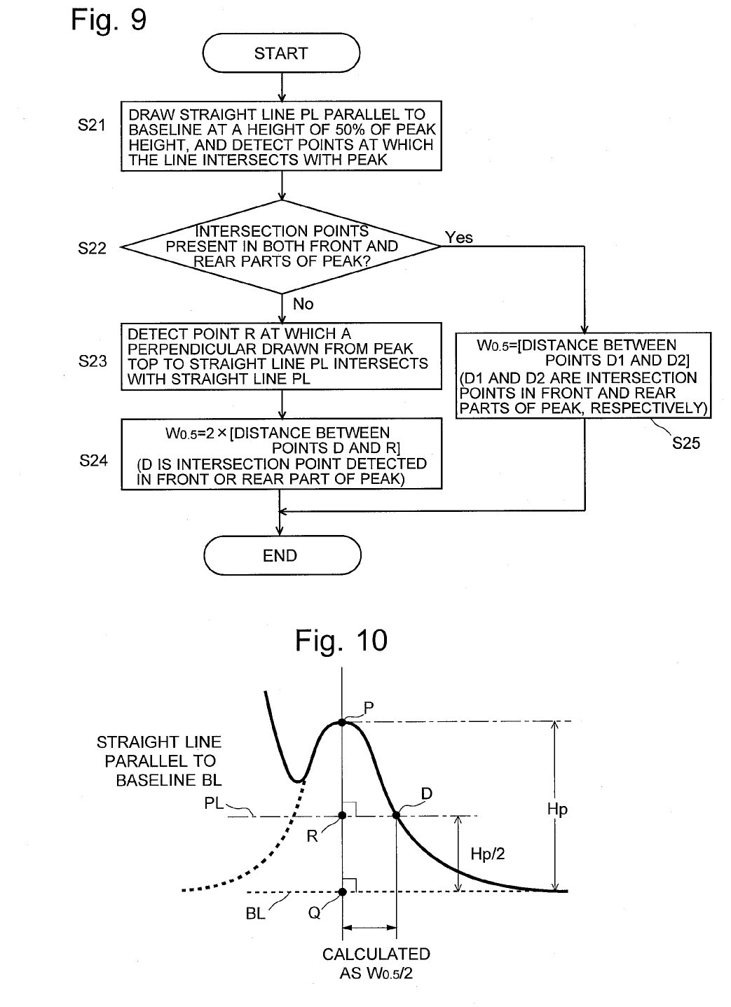

A procedure of calculating the peak width W_{0.5} at a height of 50% of the peak height according to the method of the Japanese Pharmacopeia is hereinafter described by means of the flowchart of FIG. 9, with reference to the illustrative diagrams of FIGS. 2 and 10.

In the second mode of the peak width calculation process according to the present embodiment, a straight line PL parallel to the baseline BL of the target peak is drawn at a height of 50% of the height of the peak top P from the baseline BL, and the points at each of which this line PL intersects with the peak are detected (Step S21). Subsequently, it is determined whether or not the intersection points detected in Step S21 have been obtained in both the front and rear parts of the peak (Step S22).

In Step S22, when the intersection point of the peak and the straight line PL exists in both the front and rear parts of the peak, the peak width W_{0.5} is calculated by determining the distance between the two intersection points D1 and D2, as shown in FIG. 2 (Step S25).

On the other hand, as in the case of FIG. 10, when the intersection point in one of the front and rear parts of the peak has not been located in the second mode of the peak width calculation process according to the present embodiment, a point R at which a perpendicular drawn from the peak top P to the straight line PL intersects with this line PL is located (Step S23). Then, the distance between the intersection point detected in Step S21 (the point D in the example of FIG. 10) and the intersection point R is calculated, and a value obtained by doubling this distance is adopted as the peak width W_{0.5} (Step S24).

Thus, even if one of the intersection points of the target peak and the straight line PL cannot be detected, the peak width W_{0.5} can be calculated by the processes performed in Steps S23 and S24.

It should be noted that the previously described embodiment is a mere example of the present invention and can evidently be changed or modified appropriately within the spirit of the present invention in aspects other than the previously described ones. For example, although the second mode of the peak width calculation process in the previously described embodiment was the case of calculating the peak width at a height of 50% of the height of the peak top from the baseline, it is possible to more generally calculate a peak width at a height of M % (0<M<100) by a similar procedure.

In a method described in the Japanese Unexamined Patent Application Publication No. 2004-184148, the points on the peak at a height equal to 1/e^{0.5} times the peak height from the baseline are defined as virtual inflection points, and the distance between the two points at which the tangents to the peak at the virtual inflection points in the front and rear parts of the peak respectively intersect with the baseline is calculated as the peak width W. In such a calculation method, the detection of the virtual inflection points and the determination on the detected virtual inflection points can be performed by a process similar to Steps S21 and S22 in FIG. 9, and the calculation of the peak width after the determination can be performed by a process similar to Steps S13-S17 in FIG. 7.

### EXPLANATION OF NUMERALS

11 . . . Syringe
12 . . . Sample Vaporization Chamber
13 . . . Carrier Gas Passage
14 . . . Column
15 . . . Detector
16 . . . Data Processing System
161 . . . Chromatogram Creator
162 . . . Baseline Determiner
163 . . . Peak Top Detector
164 . . . Peak Width Calculator

1. A data processing system for a chromatograph, comprising:
a) a baseline determiner for determining a baseline of a chromatogram;
b) a peak top detector for detecting a peak top of the chromatogram; and
c) a peak width calculator for calculating a peak width of a peak by detecting an inflection point in a front part or a rear part of the peak divided at the peak top, by detecting an intersection point at which a tangent to the peak at the inflection point intersects with the baseline as well as an intersection point at which a perpendicular drawn from the peak top to the baseline intersects with the baseline, by determining the distance between the two intersection points, and by calculating, as the peak width of the peak, a value which equals two times the aforementioned distance.

2. The data processing system for a chromatograph according to claim 1, wherein, when it is possible to detect a true inflection point of the peak in both the front part and the rear part of the peak, the peak width calculator detects two intersection points at which the tangents to the peak at the two inflection points respectively intersect with the baseline, and calculates the distance between the two intersection points as the peak width.

3. The data processing system for a chromatograph according to claim 2, wherein a determination on whether or not the detected inflection points are true inflection points of the peak is performed based on whether or not the inflection points are located within a predetermined range from a height equal to 1/e^{0.5} times a height of the peak top from the baseline, where e is a base of natural logarithm.

4. The data processing system for a chromatograph according to claim 1, wherein the inflection point is a virtually detected point at which a straight line drawn parallel to the baseline at a height equal to 1/e^{0.5} times a height of the peak top from the baseline intersects with the peak.

5. A data processing system for a chromatograph, comprising:
a) a baseline determiner for determining a baseline of a chromatogram;
b) a peak top detector for detecting a peak top of the chromatogram; and
c) a peak width calculator for calculating a peak width of a peak by drawing a straight line parallel to the baseline at a height of M-% of a height of the peak top from the baseline, by detecting an intersection point at which the straight line intersects with the peak in a front part or a rear part of the peak divided at the peak top, by detecting an intersection point at which a perpendicular drawn from the peak top to the straight line intersects with the straight line, by determining the distance between the two intersection points, and by calculating, as the peak width of the peak, a value which equals two times the aforementioned distance.

6. The data processing system for a chromatograph according to claim 5, wherein, when it is possible to detect intersection points of the peak and the straight line in both the front part and the rear part of the peak, the peak width calculator calculates the distance between the two intersection points as the peak width.
